(19) United States
(12) Patent Application Publication
(10) Pub. No.: US 2023/0320655 A1
(43) Pub. Date: Oct. 12, 2023

(54) Systems and Methods for Using Triaxial Accelerometer Data for Sleep Monitoring

(71) Applicant: The Board of Trustees of the Leland Stanford Junior University, Stanford, CA (US)

(72) Inventors: Todd Coleman, Stanford, CA (US); Sandya Subramanian, Stanford, CA (US)

(73) Assignee: The Board of Trustees of the Leland Stanford Junior University, Stanford, CA (US)

(21) Appl. No.: 18/299,595

(22) Filed: Apr. 12, 2023

Related U.S. Application Data
(60) Provisional application No. 63/362,879, filed on Apr. 12, 2022.

(51) Int. Cl. A61B 5/00

(52) U.S. Cl. CPC A61B 5/4809; A61B 5/7267; A61B 2562/0219

(57) ABSTRACT

A device may obtain triaxial accelerometer training data for a plurality of subjects. A device may define a number of feature vectors for each time interval and each subject of the triaxial accelerometer data. A device may cluster the feature vectors of the triaxial training data into a number of clusters to obtain a cluster assignment for each of the feature vectors of each subject. A device may fit a hidden Markov model to the to the triaxial accelerometer training data cluster assignments. A device may identify at least one state for the subjects based on the cluster assignments.

### CROSS-REFERENCE TO RELATED APPLICATIONS

The current application claims priority to U.S. Provisional Patent Application No. 63/362,879 filed Apr. 12, 2022, the disclosure of which is incorporated herein by reference in its entirety for all purposes.

### GOVERNMENT SUPPORT

This invention was made with Government support under contract FELLOWSHIP NS124835 awarded by the National Institutes of Health. The Government has certain rights in the invention.

### FIELD OF THE INVENTION

The invention generally relates to monitoring of patient activity. In particular is relates to automated monitoring of patient's activity at home.

### BACKGROUND

Actigraphy, or the non-invasive study of human activity—rest cycles, is a field of study of growing importance as ambulatory and at-home monitoring of patients becomes more popular. It has the potential to revolutionize clinical trials, sleep studies, and even patient monitoring of recovery after injury or surgery.

There are several existing algorithms to classify sleep and wake from actigraphy data, most prominently the Cole-Kripke algorithm. This algorithm uses a specific formula to classify time windows as wake or sleep. However, this algorithm, like several other validated algorithms, relies on a proprietary measure of activity.

### SUMMARY OF THE INVENTION

In some embodiments, the techniques described herein relate to a process for training a classifier to determine sleep and or wake status of a subject based on triaxial accelerometer data. In an embodiment, the process including: obtaining triaxial accelerometer training data for a plurality of subjects; defining a number of features for each time interval and each subject of the triaxial accelerometer data; clustering the features of the triaxial training data into a number of clusters to obtain a cluster assignment for each of the features of each subject; fitting a hidden Markov model to the to the triaxial accelerometer training data cluster assignments; and identifying at least one state for the subjects based on the cluster assignments.

In another embodiment, the features are normalized with a z-score on a subject-by-subject basis.

In a further embodiment, each time interval is around 1 minute.

In yet another embodiment, the number of clusters is around 10 clusters.

In an additional embodiment, fitting a hidden Markov model to the to the triaxial accelerometer training data cluster assignments include using a Baum Welch algorithm.

In another further embodiment, the at least one state indicates a physical condition of at least one subject.

In yet another embodiment, the at least one state indicates a sleeping condition of at least one subject.

In another embodiment again, the at least one state indicates a waking condition of at least one subject.

In yet another embodiment again, identifying at least one state for the subjects based on the cluster assignments is based on frequency distributions of the clustering assignments.

In a yet further embodiment, clustering assignments are captured as emission probabilities of different states in the hidden Markov model.

In still yet another embodiment, the at least one state is identified as a sleep state when the at least one state occurs beyond a threshold proportion during ground truth sleep.

In still yet another embodiment again, the at least one state is identified as a sleep state when the at least one state occurs more than 60% of the time during ground truth sleep.

In an additional further embodiment, the features include at least one item selected from a list including: standard deviation of each axis, a coefficient of variation of each axis, a range along each axis, an interquartile range of each axis, a number of small peak (local maxima) in each axis, a number of medium peaks in each axis, and a number of large peaks in each axis.

In a yet still additional embodiment, the process is validated using leave-one-subject-out cross-validation.

In another additional embodiment again, the clustering the features of the triaxial training data is performed using K-means clustering.

In some embodiments, the techniques described herein relate to a process for classifying triaxial accelerometer data to identify subject sleep states. In an embodiment, the process including: obtaining triaxial accelerometer data for a subject; mapping subject features from the triaxial accelerometer data onto at least one cluster based on a closest centroid; determining a most likely trajectory of hidden Markov model states based on the mapping of the subject features onto the at least one cluster; and classifying a state of the subject in a time series.

In another embodiment, the process further including identifying the state of the subject for each time in the time series, as either asleep or awake.

In yet another embodiment, awake periods below a threshold duration in-between periods of sleep are defined as sleep.

In another embodiment again, awake periods less than around 15 minutes in duration in-between periods of sleep are defined as sleep.

In a further embodiment, a state is classified as sleep only when the hidden Markov model predicts probability of a sleep state was at least 0.95.

In a further embodiment again, a process further including discarding any periods of sleep under a duration.

In a yet further embodiment again, a process including discarding any periods of sleep under one hour in duration.

In still another embodiment, the triaxial accelerometer data is obtained from a wearable device.

In still yet another embodiment, the triaxial accelerometer data is obtained from a mobile device.

In still yet further embodiment, transient occurrences of sleep cluster labels outside of sleep are categorized into other states by the hidden Markov model.

In still another embodiment again, a Viterbi algorithm is used to determine the most likely trajectory of hidden Markov model states.

In still yet another embodiment again, the features are feature vectors.

### BRIEF DESCRIPTION OF THE DRAWINGS

The description will be more fully understood with reference to the following figures, which are presented as exemplary embodiments of the invention and should not be construed as a complete recitation of the scope of the invention.

FIG. 1 conceptually illustrates an example process for training a triaxial accelerometer sleep and/or wake state classifier.

FIG. 2 depicts examples of features for use in classifying sleep and/or wake states.

FIG. 3 conceptually illustrates an example process for obtaining a sleep and/or wake state classification based on a triaxial accelerometer sleep and/or wake state classifier.

FIG. 4 shows example of results from performing a leave-one-subject-out cross-validation for subjects.

FIG. 5 shows example cluster centroids for clusters strongly associated with sleep states.

FIG. 6 shows an example summary comparison of cross-validated results between a tri-axial accelerometer algorithm method as described herein and the Cole-Kripke algorithm.

FIG. 7A through G depict example raw data, Cole-Kripke sleep predictions, clustering labels, and cross-validated sleep predictions from described methods for each subject among a number of subjects.

FIG. 8 conceptually illustrates an example computing device that can execute processes described herein.

### DETAILED DESCRIPTION

One of the most important applications of actigraphy is to understand natural sleep-wake patterns, especially since these are nearly impossible to truly replicate in a clinical or laboratory setting. Several embodiments of the invention are capable of this. With actigraphy, it can be possible to quantify the duration, timing, and/or quality of sleep experienced at home using various algorithms (e.g., various automated algorithms).

Several embodiments of algorithms can identify autonomic and gastric myoelectric biomarkers from throughout the day. These biomarkers can be used to differentiate patients with gastroparesis, diabetics without gastroparesis, and healthy controls, and can providing insight into etiology.

Various devices (e.g., wearable device) can extract information about physiologic signals that may be predictive of when sleep events (e.g., sleep events such as apneas and/or hypopnea) occur. Combining these pieces of information with the duration of time an individual has slept can give rise to determination of sleep apnea severity (e.g. construction of the apnea hypopnea index).

In accordance with some embodiments of the invention actigraphy to identify sleep-wake patterns can be useful for at-home diagnosis of sleep apnea. One of the parameters in identifying sleep apnea could be a determination of the duration of time the patient was sleeping (e.g., as provided by various embodiments described herein).

### Automatic Classification

In accordance with various embodiments of the invention, features can be provided to a classifier to determine if those features can be used to automatically delineate subjects based on specific statuses (e.g., sleep-wake). In some embodiments, a set features can be extracted from a subject specific data set to classify subject status (e.g., sleep-wake).

Determining Sleep and Wake from Actigraphy

Starting from the 24-hour triaxial accelerometer information for each recording as well as the manual annotations, several embodiments are capable of inferring accurate sleep and wake times. For numerous embodiments, triaxial accelerometer information can be used to infer sleep and wake time such that only slight deviations from the manually annotated times.

The Cole-Kripke algorithm uses a specific formula to classify time windows as wake or sleep. However, one major drawback of this algorithm is that it, like several other validated algorithms, relies on a proprietary measure of activity. The proprietary measure can be output only a small proportion of devices. This limits the usability of these algorithms. On the other hand, all smart devices have built-in triaxial accelerometers that can also be used to track activity cycles in a widely available format. In several embodiments data from triaxial accelerometers can be used to track activity cycles. Tracking activity cycles can include tracking waking and/or sleeping cycles.

Actigraphy allows for the remote monitoring of subjects' activity for clinical and research purposes. However, most standard methods are built for proprietary measures from specific devices that are not widely used. In many embodiments, an algorithm (e.g., a classification algorithm) can perform classification of sleep and awake using an amount (e.g., a single day) of triaxial accelerometer data. This accelerometer data can, in accordance with embodiments of the invention, be acquired from many smart devices. A classification algorithm can consist of two stages. The stages can be clustering and hidden Markov modeling. Various embodiments of classification algorithms as described herein outperform standard algorithms in sensitivity (94%), specificity (93%), and overall accuracy (93%) across seven subjects. Methods and/or systems as described herein can help automate actigraphy analyses at scale using widely available technology using even a single day's worth of data.

Furthermore, automated monitoring of patients' activity at home can help track recovery trajectories after surgery and injury, disease progression, and treatment response. In addition, actigraphy traditionally often relied on data across multiple days or weeks to characterize routines and activity—rest cycles of a single person. While this may be possible for those who follow strict routines, reliance on longitudinal data collection to establish sleep patterns is often not possible for those with erratic sleeping patterns or changing schedules, or in cases of shorter duration studies. In many embodiments, an automated algorithm can be capable of classifying sleep and wake robustly using triaxial accelerometer data on a single day basis. This algorithm can be beneficial to broaden the usability of actigraphy for clinical, research, and consumer device purposes.

In an example study, a single 24-hour session of triaxial accelerometer data was collected from 7 subjects with accompanying manual annotations of sleep and wake. In various embodiments, a two-part algorithm using K-means clustering and Hidden Markov modeling can classify sleep vs wake (e.g., classify sleep vs wake robustly across subjects on a minute-by-minute basis). Comparing the properties and dynamics of identified sleep and wake states can be performed across subjects. A Cole-Kripke algorithm used for comparison shows that several embodiments of an algorithm using triaxial accelerometer data can be both more accurate and more robust at classification than the Cole-Kripke algorithm.

The example study above included continuous 24-hour triaxial accelerometer data were recorded from 7 subjects (ages 22-70, 5 females) under protocol approved by the University of California San Diego Institutional Review Board using an OpenBCl Cyton ambulatory electrophysiological recording system. Subjects were also required to manually annotate when they went to sleep and awakened during the 24-hour period.

Several embodiments of the invention can be compared to a Cole-Kripke Algorithm. As a baseline comparison to various embodiments, the Cole-Kripke algorithm can be implemented. In some cases a previously validated algorithm was used to compute rough estimates of Actilife count values from triaxial accelerometer data. Then, the computed estimates of Actilife counts for each second could be used for the Cole-Kripke algorithm. For among several variations, a selected best variation of the Cole-Kripke algorithm can be used as a comparison method based on maximizing the average of sensitivity and specificity across all subjects. In this way it can be possible to equally weight classification of sleep and awake periods, since awake periods are far more prevalent in the data and would outweigh sleep in overall accuracy.

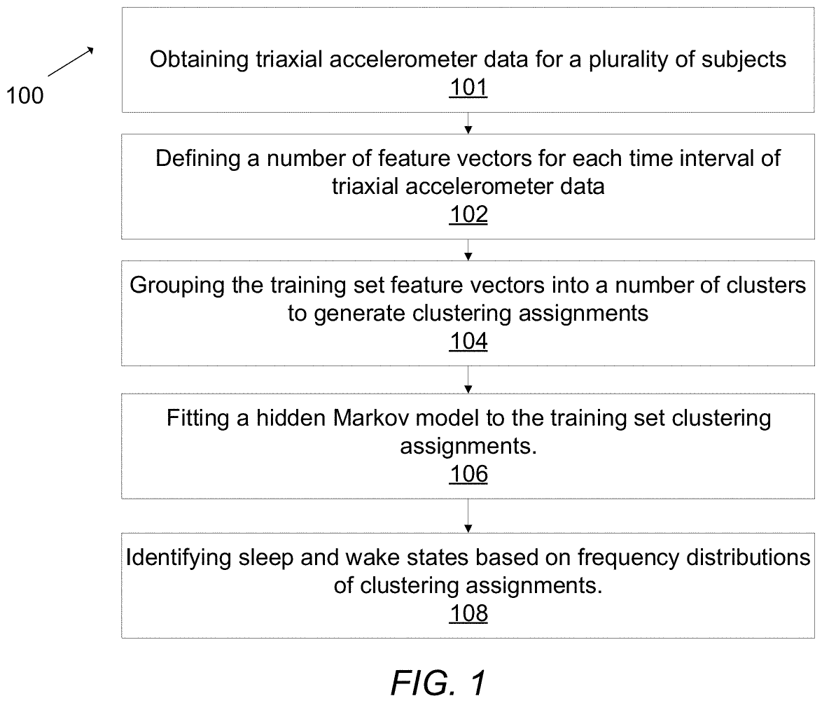

Several process can be suitable for classifying triaxial accelerometer data to determine sleep and/or wake state. An example process for training a triaxial accelerometer sleep and/or wake state classifier is conceptually illustrated in FIG. 1. A process 100 can obtain (101) triaxial accelerometer data for a plurality of subjects. The process 100 can define (102) a number of features for each time interval of triaxial accelerometer data. In several embodiments, the features can be normalized by z-score within each subject (e.g., the feature vectors can be normalized with a z-score on a subject-by-subject basis).

The process 100 can group (104) the training set feature vectors into a number of clusters (e.g., 10 clusters). Grouping training set feature vectors can result in generating clustering assignments for each of the training set feature vectors. The process 100 can fit (106) a hidden Markov model to the training set clustering assignments. Fitting a hidden Markov model to the training set clustering assignments can use Baum Welch algorithm. Using the clustering assignments, the process 100 can identify (108) sleep and wake states based on frequency distributions of the clustering assignments. Clustering assignments can be captured as the emission probabilities of different states in an HMM. In accordance with several embodiments of the invention, sleep states can be identified as states that occur a certain proportion (e.g., 60-40, 60%) during sleep as compared with awake based on the ground truth annotations. In various embodiments, the process can indicate a physical condition of at least one subject based on classifications of state.

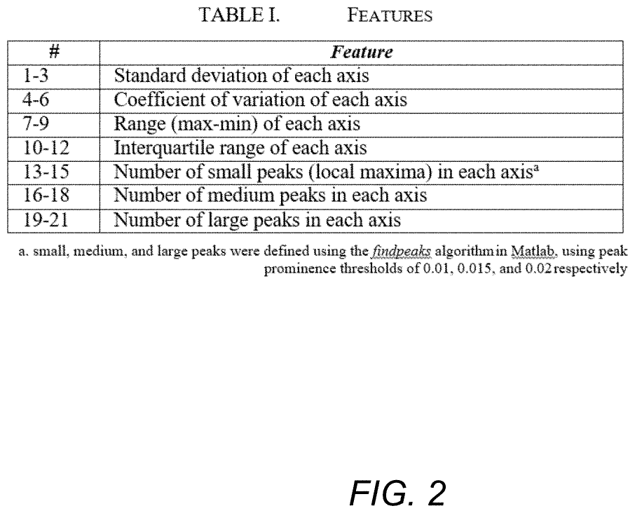

In accordance with some embodiments of the invention, features can include (as shown in FIG. 2) defined features of triaxial accelerometer data such as a standard deviation of each axis, a coefficient of variation of each axis, a range (e.g., max-min) of each axis, an interquartile range of each axis, a number of small peak (local maxima) in each axis, a number of medium peaks in each axis, and/or a number of large peaks in each axis. In many embodiments, small, medium, and large peaks can be defined using an algorithm (e.g., a findpeaks algorithm in MATLAB using peal prominence thresholds of 0.01, 0.015, and 0.02 respectively).

In various embodiments, a first step of a patient activity monitoring method can include defining a number of features (e.g., defining 21 features) for each time interval (e.g., minute) of triaxial accelerometer data. FIG. 2 depicts examples of features for use in classifying sleep and/or wake states. In many embodiments, these features were defined based on empirical observation. In several embodiments, the features can be normalized by z-score within each subject. In some embodiments the entire process can be validated using leave-one-subject-out cross-validation. For a leave-one-subject-out cross-validation, each time, one subject was designated the test subject and a remaining number (e.g., six) were used for training.

In accordance with some embodiments of the invention, in the first stage of training, the training set feature vectors can be grouped into a number of clusters (e.g., 10 clusters) using K-means clustering (e.g., with K-means++initialization in MATLAB). For some embodiments, ten was enough clusters to capture the complexity and variety of activity patterns across subjects for sleep and wake cycle tracking.

In various embodiments, a second stage of training includes fitting a hidden Markov model (HMM) to the training set clustering assignments across all subjects using the Baum Welch algorithm. While the clustering assignments themselves can be very noisy across subjects, sleep and wake are distinguishable by the frequency distributions of the specific cluster labels, which can be captured as the emission probabilities of different states in an HMM. For some embodiments, the number of states was varied between 11 and 15 for each training HMM. In each case, the sleep-associated states (‘sleep states’) were identified as states that occur more during sleep than wake in at least a 60-40 ratio using the ground truth annotations.

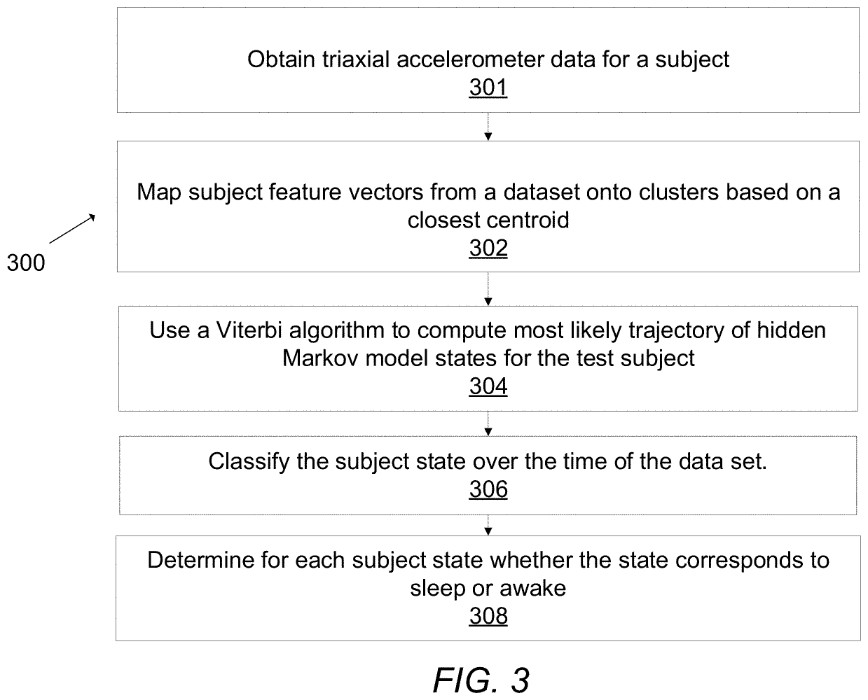

An example process for obtaining a sleep and/or wake state classification based on a triaxial accelerometer sleep and/or wake state classifier is conceptually illustrated in FIG. 3. A process 300 can use a classifier trained (e.g., using a process 200) to classify sleep and wake states based on obtained triaxial accelerometer data. A process 300 can obtain (301) triaxial accelerometer data for a subject. A process 300 can map (302) subject feature vectors from a data set onto clusters based on a closest centroid. The process 300 can then compute (304) a most likely trajectory of hidden Markov model states for the test subject (e.g., using a Viterbi algorithm). Based on the output from 304, the process 300 can classify (306) the subject's state over the time of the data set (e.g., over a time series). The process 300 can determine, for each state whether the state corresponds to sleeping or waking. Triaxial accelerometer data can be obtained from wearable devices, mobile devices, and/or other devices.

In various embodiments, awake periods of a duration (e.g., 15 minutes or less) in between periods of sleep can be defined as sleep. In some embodiments, a system can keep only sections of sleep for which the HMM predicted probability of a sleep state was at least 0.95. Of these, a process can discard any periods of sleep under 1 hour in duration to focus on overnight sleep. The remaining sleep periods can be retained in accordance with embodiments of the invention. In some embodiments, a preferred number of states for each training HMM model can be identified by maximizing the average sensitivity and specificity of sleep detection throughout.

In accordance with embodiments of the invention, during a testing phase, to compute predictions of sleep for the test subject using the trained clustering and HMM models, the features vectors of the test subject can be mapped onto the same clusters as the training data based on a closest centroid. Then a Viterbi algorithm can be used to compute the most likely trajectory of HMM states for the test subject using the optimal model fitted to the training data. In a further step, sleep and wake can be assigned using the already identified set of sleep states for each training HMM model.

A 2-stage algorithm can include using K-means clustering and hidden Markov modeling to classify sleep vs awake in 24-hour triaxial accelerometer data. In the example data this was performed for 7 subjects using 21 features.

For various embodiments, leave-one-subject-out cross-validation can be used to measure the performance of a triaxial accelerometer sleep state classifier. Not only did an example triaxial accelerometer sleep state classifier outperform the adapted Cole-Kripke algorithm for each of 7 subjects in terms of accuracy, sensitivity, and specificity, it also achieved an average accuracy of 92.99%.

In several embodiments, even though the goal of a triaxial accelerometer sleep state classifier is to distinguish between two subject conditions (e.g., sleep and awake), far more states can necessary for the classifier because of the diversity of activity patterns across subjects and the resulting variability inherent to the clustering labels returned by K-means clustering. In several embodiments, identified sleep states and associated clusters can have the expected properties of sleep, in terms of features (lowest variance, smallest range, etc.). An HMM can distinguish between differing probability distributions of the same cluster labels, so erroneous transient occurrences of sleep cluster labels outside of sleep can be accurately categorized into other states.

In the example data, Subject 6, has distinctly lower performance compared to the remaining subjects with the triaxial accelerometer sleep state classifier. This can indicate an anomaly and/or additional noise source in the raw data of Subject 6.

The ability to monitor ambulatory and/or at-home activity for clinical and research purposes can be of high value for at-home clinical trials, personalizing recovery trajectories after surgery and/or injury, and/or tracking disease progression and/or treatment response. By using triaxial accelerometer data, various embodiments can be integrated into processing pipelines with widely available smart devices in the home (e.g., tablets, phones, and/or watches). Sleep patterns of varying complexity, can in several embodiments, can be classified in a manner similar to those described throughout.

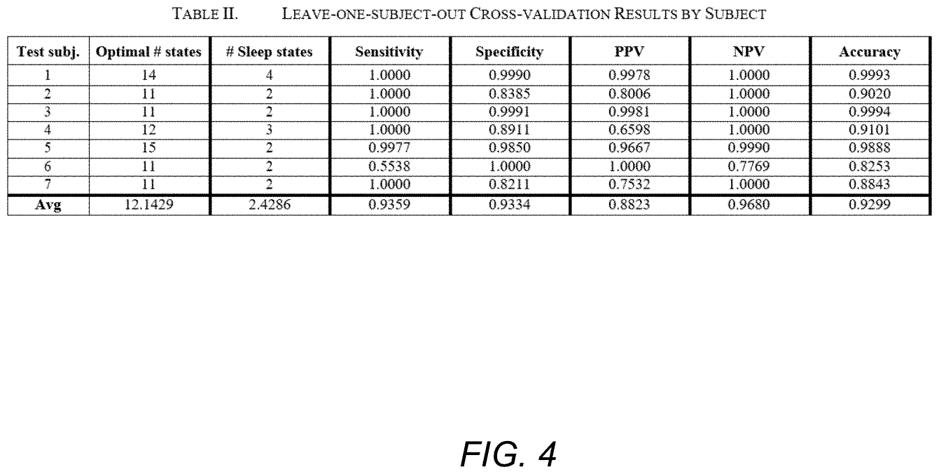

An example of results from performing a leave-one-subject-out cross-validation for subjects is shown in FIG. 4. As can be seen, different test subjects can have different cross-validation results. PPV stands for positive predictive value. NPV stands for negative predictive value.

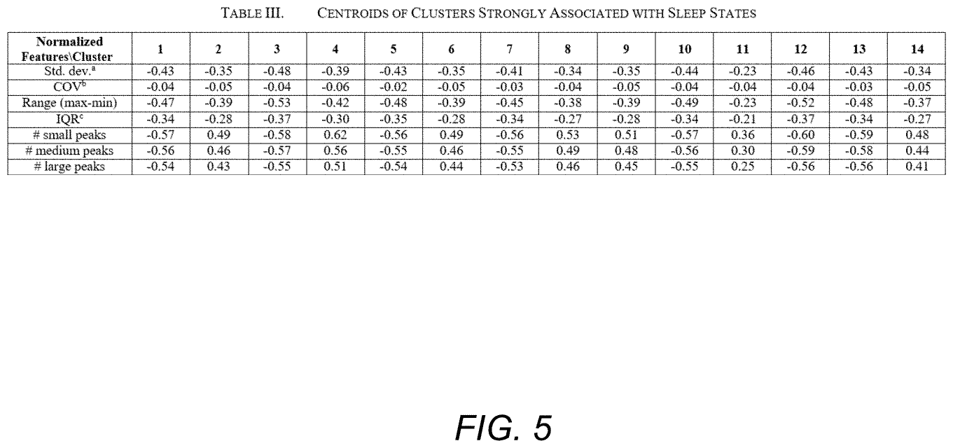

Example cluster centroids for clusters strongly associated with sleep states are shown in FIG. 5. Std. dev. is standard deviation. COV is coefficient of variation. IQR is interquartile range.

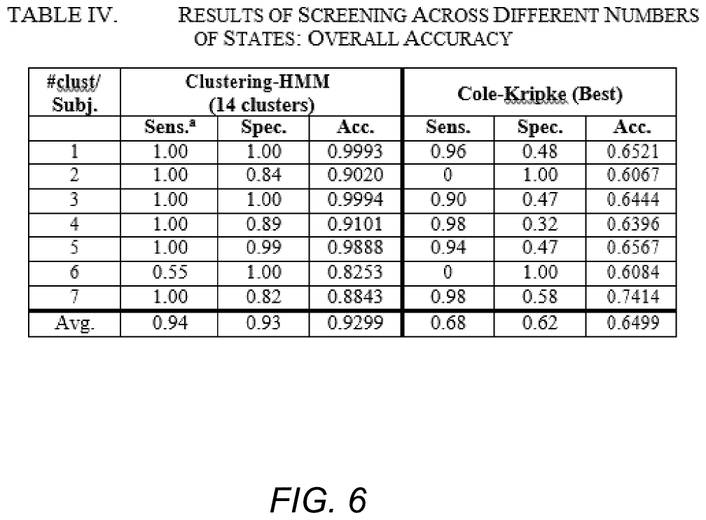

An example comparison of cross-validated results between a tri-axial accelerometer algorithm method as described herein and the Cole-Kripke algorithm is summarized in FIG. 6. In FIG. 6, Sens is sensitivity, Spec is specificity, and Acc is overall accuracy.

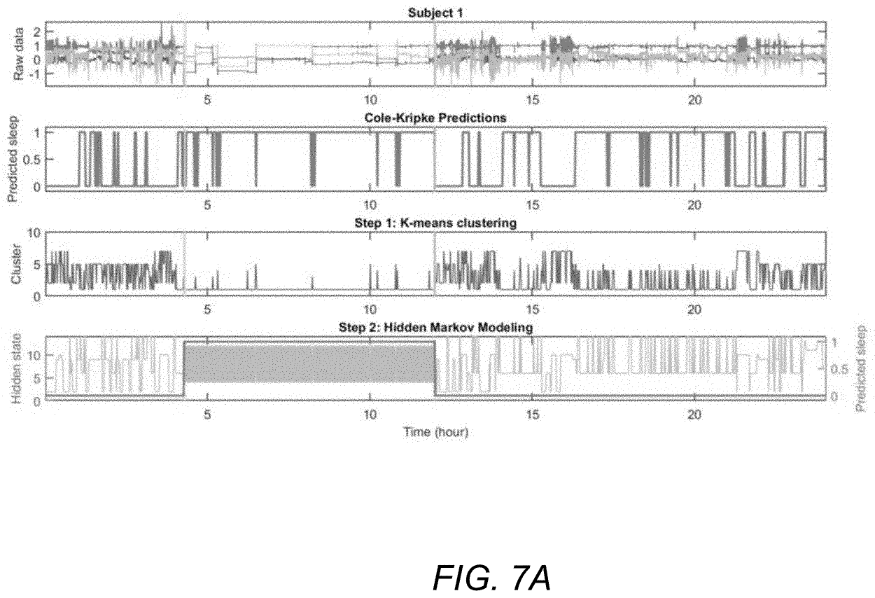

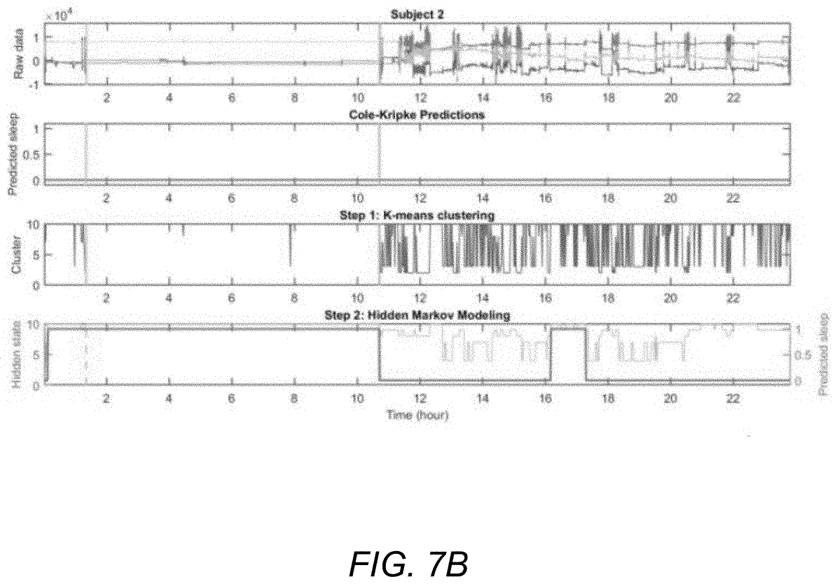

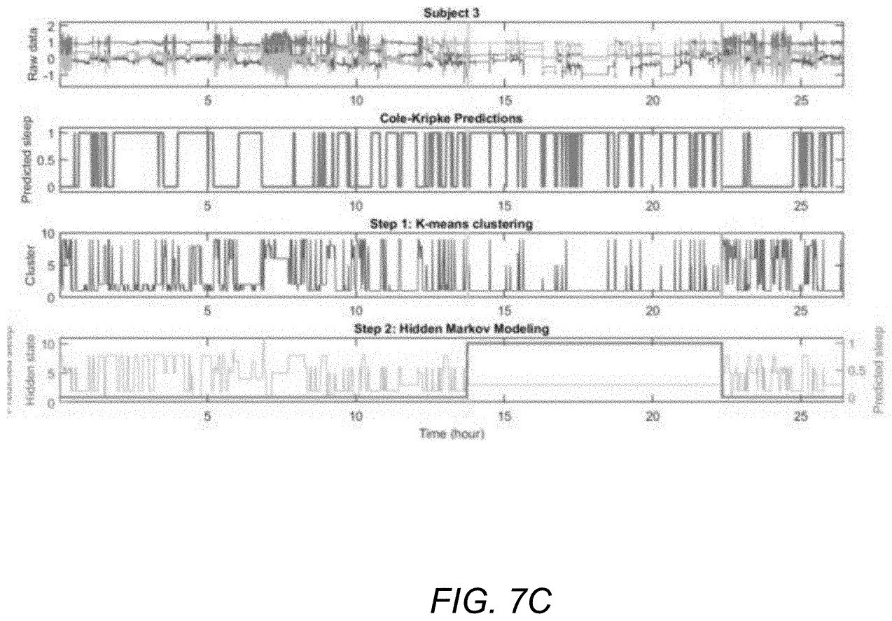

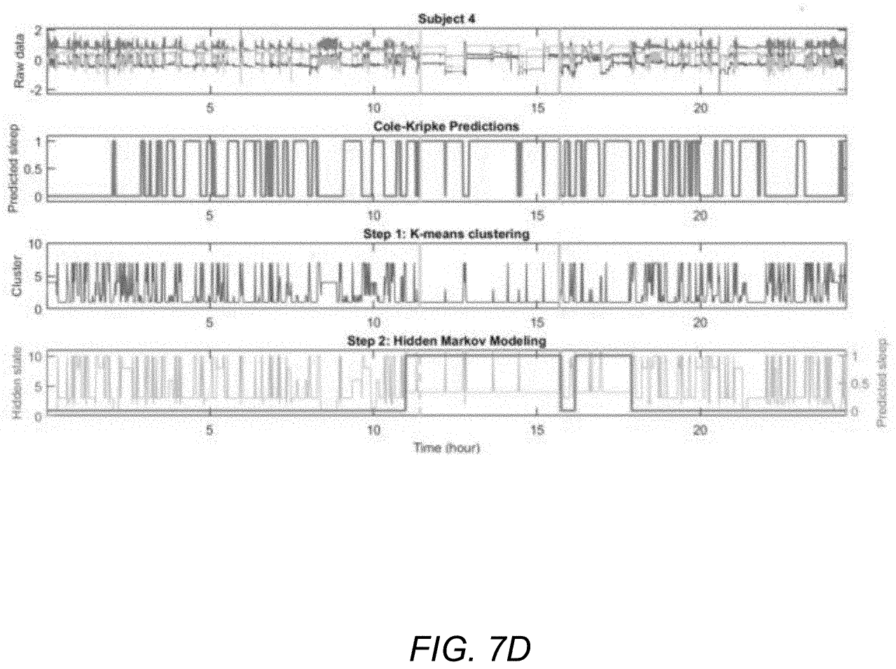

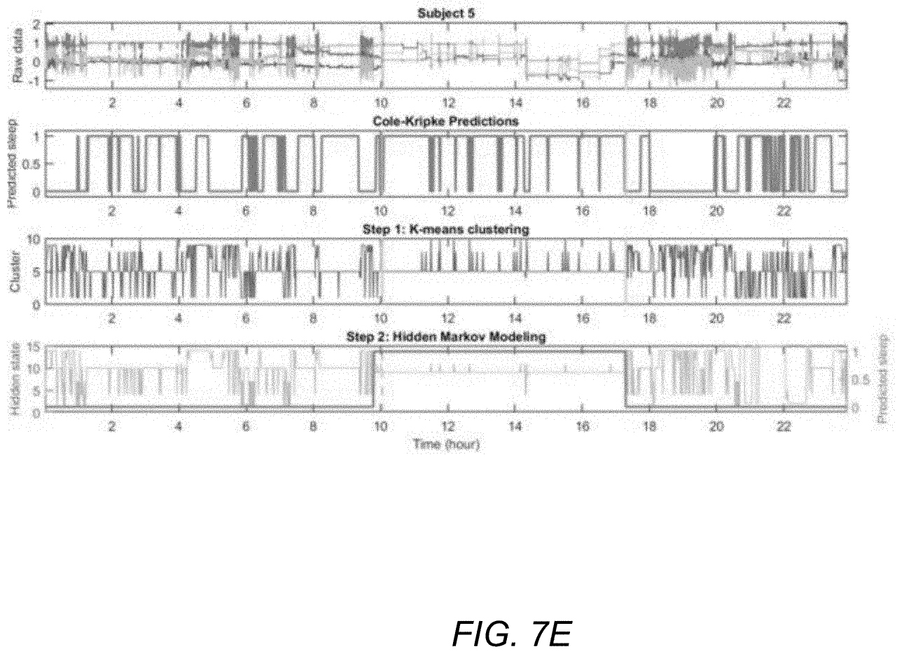

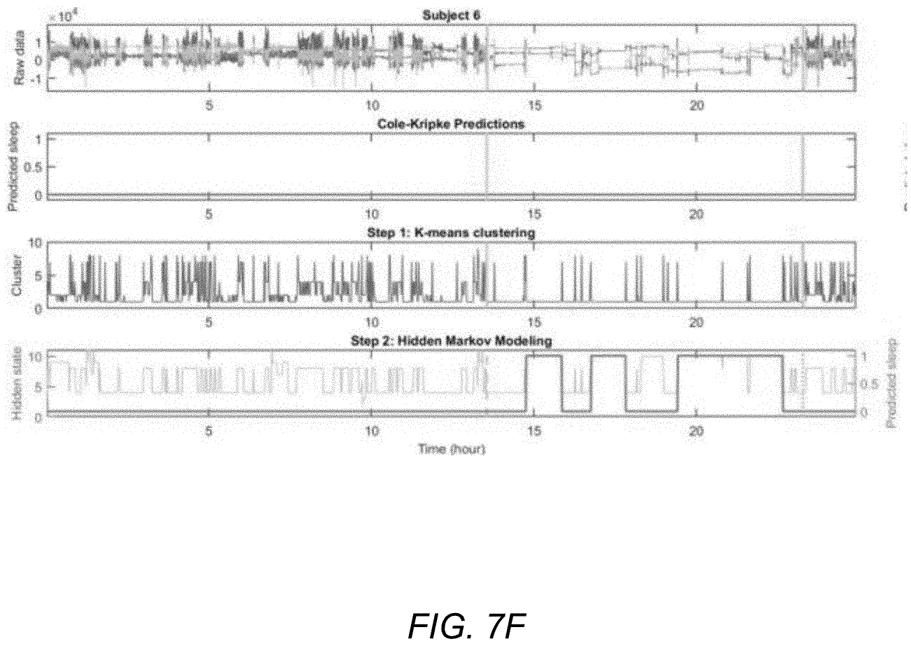

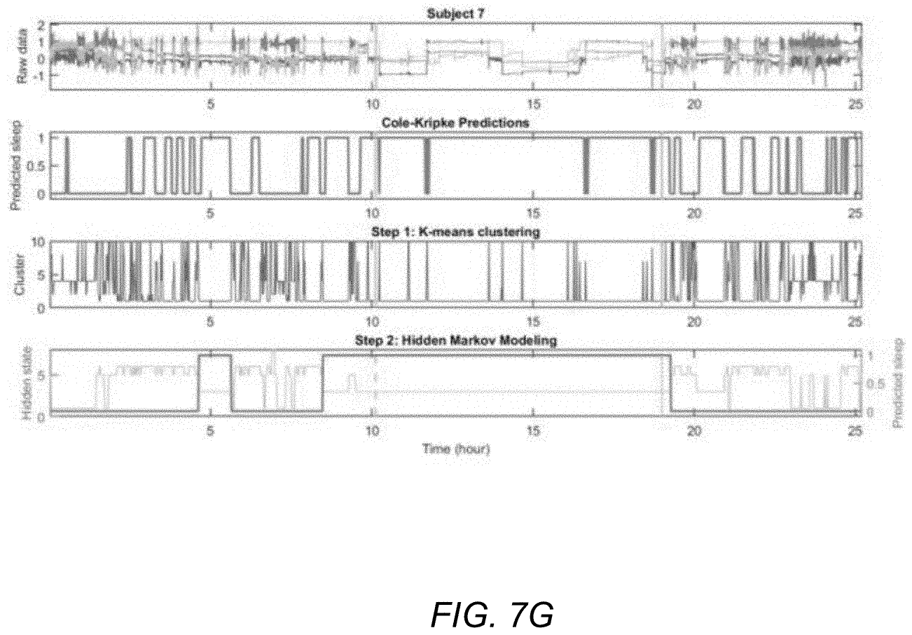

Example raw data, Cole-Kripke sleep predictions, clustering labels, and cross-validated sleep predictions from described methods for each subject among a number of subjects are shown in FIG. 7A through G. As can be seen in the figure, various embodiments are effective at determining sleep states based on triaxial accelerometer data. In the example data, the average number of states across subjects was 12.1 with 11 the most popularly occurring (e.g., as seen in FIG. 4). In cross-validation, an example sleep state algorithm achieved an average accuracy of 93% across subjects, with an average sensitivity of 93.6% and an average specificity of 93.3% (e.g., FIG. 4 and FIG. 6). All subjects achieve over 82% accuracy, with 6 out 7 achieving over 90% accuracy. In the example, Five out of 7 subjects achieve 100% sensitivity, and 4 out of 7 achieve at least 98% specificity. The average positive predictive value was 88.2%, with 4 out of 7 subjects achieving higher than 96%. The average negative predictive value was 96.8%, with 6 out of 7 subjects achieving around 100%.

Continuing the explication of the example data shown in FIG. 7A through G, the average number of sleep states across subjects was 2.4. Sleep states were typically associated with a single cluster label with an emission probability of over 90%. The feature vectors of the associated sleep clusters have the lowest normalized values for most features, indicating lowest variance of activity. In this example data, subject 6 does not perform as well as the others, achieving only 82.5% accuracy and 55.3% sensitivity. This may be due to detecting sleep only in smaller disjoint segments. While the raw data are clearly different during sleep compared to wake, the error may come from slight changes in cluster label distribution during sleep.

In FIG. 7A through G, various example subjects are shown with associated data. Each of FIG. 7A through G showing from top to bottom: (1) the raw data, (2) Cole-Kripke algorithm predictions (sleep is 1), (3) K-means clustering labels, and (4) HMM state labels with final sleep-wake labels after re-scoring. In all plots, the cyan and green vertical lines mark the onset and end of sleep as ground truth.

In contrast, and as shown in FIG. 7A through 7G, the Cole-Kripke algorithm achieves an average overall accuracy of only 65%, an average sensitivity of 68%, and an average specificity of 62% across subjects (FIG. 4). The error may derives mostly from the occurrence of false positives, except in the case of 2 subjects for whom the algorithm incorrectly predicts no sleep. The example triaxial accelerometer data driven algorithm outperforms the Cole-Kripke algorithm for all subjects in terms of overall accuracy, sensitivity, and specificity. This is mainly due to the reduction in false positives (except for Subjects 2 and 6).

In accordance with various embodiment of the invention, triaxial accelerometer data can be combined with data from other sensors (e.g., sensors for nasal and/or oronasal airflow, EEG, EOG, submental EMG, ECG, TcCO2, EMG, pulse oximetry, and/or chest and abdominal inductance plethysmography EMG). Several embodiments can provide uses for diagnoses of obstructive sleep apnea in habitually snoring children. The data can be supplied by a wearable device. The wearable device can have various integrated sensors.

### Computer System

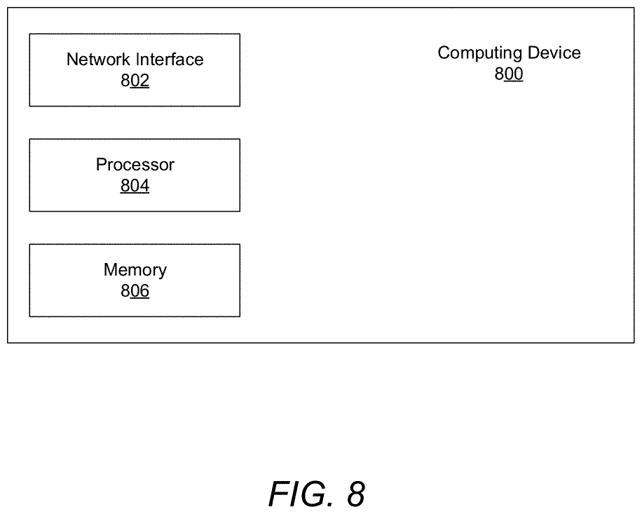

An example computing device that can execute processes described herein is conceptually illustrated in FIG. 8. A computing device 800 can include a network interface 802, a processor 804, and a memory 806. The memory 806 can contain instructions to cause the processor 804 to execute processes and instructions as described elsewhere herein. Computer systems can have non-transitory memory. Non-transitory memory can be used to store instructions for executing processes.

### Doctrine of Equivalents

While the above description contains many specific embodiments of the invention, these should not be construed as limitations on the scope of the invention, but rather as an example of one embodiment thereof. It is therefore to be understood that the present invention may be practiced in ways other than specifically described, without departing from the scope and spirit of the present invention. Thus, embodiments of the present invention should be considered in all respects as illustrative and not restrictive. Accordingly, the scope of the invention should be determined not by the embodiments illustrated, but by the appended claims and their equivalents.

What is claimed is:

1. A process for training a classifier to determine sleep and or wake status of a subject based on triaxial accelerometer data, the process comprising:
obtaining triaxial accelerometer training data for a plurality of subjects;
defining a number of features for each time interval and each subject of the triaxial accelerometer data;
clustering the features of the triaxial training data into a number of clusters to obtain a cluster assignment for each of the features of each subject;
fitting a hidden Markov model to the to the triaxial accelerometer training data cluster assignments; and
identifying at least one state for the subjects based on the cluster assignments.

2. The process of claim 1, wherein each time interval is around 1 minute.

3. The process of claim 1, wherein the number of clusters is around 10 clusters.

4. The process of claim 1, wherein fitting a hidden Markov model to the to the triaxial accelerometer training data cluster assignments comprise using a Baum Welch algorithm.

5. The process of claim 1, wherein the at least one state indicates a sleeping condition of at least one subject.

6. The process of claim 1, wherein identifying at least one state for the subjects based on the cluster assignments is based on frequency distributions of the clustering assignments.

7. The process of claim 1, wherein clustering assignments are captured as emission probabilities of different states in the hidden Markov model.

8. The process of claim 1, wherein the features include at least one item selected from a list including: standard deviation of each axis, a coefficient of variation of each axis, a range along each axis, an interquartile range of each axis, a number of small peak (local maxima) in each axis, a number of medium peaks in each axis, and a number of large peaks in each axis.

9. The process of claim 1, wherein the clustering the features of the triaxial training data is performed using K-means clustering.

10. A process for classifying triaxial accelerometer data to identify subject sleep states, the process comprising:
obtaining triaxial accelerometer data for a subject;
mapping subject features from the triaxial accelerometer data onto at least one cluster based on a closest centroid;
determining a most likely trajectory of hidden Markov model states based on the mapping of the subject features onto the at least one cluster; and
classifying a state of the subject in a time series.

11. The process of claim 10, further comprising identifying the state of the subject for each time in the time series, as either asleep or awake.

12. The process of claim 10, wherein awake periods below a threshold duration in-between periods of sleep are defined as sleep.

13. The process of claim 10, wherein awake periods less than around 15 minutes in duration in-between periods of sleep are defined as sleep.

14. The process of claim 10, wherein a state is classified as sleep only when the hidden Markov model predicts probability of a sleep state was at least 0.95.

15. The process of claim 10, further comprising discarding any periods of sleep under a duration.

16. The process of claim 10, further comprising discarding any periods of sleep under one hour in duration.

17. The process of claim 10, wherein the triaxial accelerometer data is obtained from a wearable device.

18. The process of claim 10, wherein the triaxial accelerometer data is obtained from a mobile device.

19. The process of claim 10, wherein transient occurrences of sleep cluster labels outside of sleep are categorized into other states by the hidden Markov model.

20. The process of claim 10, wherein a Viterbi algorithm is used to determine the most likely trajectory of hidden Markov model states.
